(19) United States
(12) Patent Application Publication
(10) Pub. No.: US 2007/0015688 A1
(43) Pub. Date: Jan. 18, 2007

(54) Method for screening for inhibitors of alzheimer's disease

(76) Inventors: Kevin Barnham, Coburg (AU); Michael Parker, Newport (AU); Robert Cappai, Kew (AU)

Correspondence Address: Leopold Presser;Scully Scott Murphy & Presser, 400 Garden City Plaza, Garden City, NY, 11530, US

(21) Appl. No.: 10/450,549

(22) PCT Filed: Dec. 12, 2001
(86) PCT No.: PCT/AU01/01603

§ 371 Date: Nov. 19, 2003

(30) Foreign Application Priority Data
Dec. 12, 2000 (AU) PR 2024

(51) Int. Cl. A61K 38/16; G01N 33/53

(52) U.S. Cl. 514/6; 435/7.1

(57) ABSTRACT

This invention relates to compounds which have the ability to act as agonists for the binding of divalent copper ions to amyloid precursor protein (APP) and to methods of identifying such compounds by using the three-dimensional structure of the copper-binding domain of APP.

### FIELD OF THE INVENTION

This invention relates to compounds which have the ability to act as agonists for the binding of copper ions to amyloid precursor protein, and to methods of identifying such compounds.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Alzheimer's disease is characterised by the presence of distinctive lesions in the patient's brain. These brain lesions include abnormal intracellular filaments, called neurofibrillary tangles, and extracellular deposits of amyloid in senile, or amyloid, plaques. Amyloid deposits are also present in the walls of cerebral blood vessels of Alzheimer's patients. The major constituent of amyloid plaques has been identified as a 4 kilodalton peptide (39-43 residues) called β-amyloid peptide (Aβ) (Glenner and Wong, 1984). Diffuse deposits of Aβ peptides are frequently observed in normal adult brains, whereas Alzheimer's disease brain tissue is characterised by more compacted, dense-core β-amyloid plaques. These observations suggest that Aβ deposition precedes, and contributes to, the destruction of neurons which occurs in Alzheimer's disease. In further support of a direct pathogenic role for Aβ, β-amyloid has been shown to be toxic to mature neurons, both in culture and in vivo (Yanker, 1996).

Natural Aβ is derived from proteolysis from a much longer protein, known as the amyloid precursor protein (APP) (Kang et al., 1987). The APP gene maps to chromosome 21, and it is thought that this explains the β-amyloid deposition which is seen at an early age in individuals with Down's syndrome, which is caused by trisomy of chromosome 21; the pattern of deposition of β-amyloid in Down's syndrome is similar to that seen in Alzheimer's disease. However, the physiological function of APP has not yet been established.

There are three main isoforms of APP, which respectively contain 695, 751 and 770 amino acids (Hardy, 1997). These forms are generally referred to as APP695, APP751 and APP770 respectively. APP undergoes consecutive proteolytic degradation by β-secretase and γ-secretase, forming Aβ. In contrast, APP degradation within the Aβ domain by α-secretase prevents the formation of amyloid, and results in the release of the p3 peptide, consisting of Aβ residues 17 to 40 or 17 to 42. Degradation of APP by α-secretase or β-secretase results in soluble APP fragments, referred to as sAPPα and sAPPβ, which represent the APP ectodomains which are released into the extracellular space. This degradation is frequently referred to as “APP processing”.

The Aβ peptides then undergo aggregation to produce the toxic β-sheet structures found in extracellular deposits in Alzheimer's disease and Down's syndrome. Recent data suggest that the aggregated peptide has redox properties and can generate reactive oxygen species, which attack enzymes and possibly cell membranes, causing neurotoxicity (Bush, 2000).

APP expression modulates copper homeostasis, since APP−/− mice have elevated copper levels in the liver and cerebral cortex when compared to APP+/+ mice (White et al., 1999b). In addition, elevated copper concentrations reduce Aβ production and increase secretion of APP in a cell-line transfected with human APP cDNA (Borchardt et al., 1999). This effect is modified by zinc, or by zinc and copper chelators (Borchardt et al., 2000). Therefore APP has an important role in modulating cellular copper metabolism in certain tissues, including the brain. Moreover, neurons expressing wild-type APP (APP+/+) are significantly more sensitive to copper toxicity than are APP-deficient neurons (APP−/−) (White et al,. 1999a), and interaction of APP-Cu(I) species with hydrogen peroxide can result in Cu(I) oxidation to Cu(II) and APP fragmentation (Multhaup et al., 1998). Therefore alterations to APP and/or copper metabolism, such as those found in Alzheimer's disease, could potentially result in increased APP-Cu(I)-mediated generation of reactive oxygen intermediates and increased oxidative stress, as well as altered APP processing to Aβ (Borchardt et al., 1999; Cherny et al., 1999). However, copper neurotoxicity mediated by full length APP has not hitherto been directly demonstrated in vivo or in vitro.

Although the fundamental pathology, genetic susceptibility and biology associated with AD are becoming clearer, a rational chemical and structural basis for developing effective drugs to prevent or cure the disease remains elusive. While the genetics of Alzheimer's disease indicate that the metabolism of Aβ is intimately associated with the pathogenesis of the disease as indicated above, drugs for the treatment of Alzheimer's disease have so far focused on “cognition enhancers”, which do not address the underlying disease processes. These drugs have met with only limited success. Thus there is a need in the art for additional agents for treatment of Alzheimer's disease.

International Patent Application No. PCT/AU92/00610 by The University of Melbourne shows that chelating agents which have the ability to bind di- or trivalent metal ions such as Cu²⁺, Zn²⁺ or Fe³⁺ are able to dissolve amyloid deposits from brains of patients with Alzheimer's disease. The inventors proposed the hypothesis that large metal ions were required in order to induce aggregation of Aβ into amyloid fibrils. These findings have also been used as the basis for assays for candidate pharmaceutical agents for the achievement of Alzheimer's disease; see for example International patent application No. PCT/US94/11928, PCT/US98/04683, U.S. patent application Ser. No. 09/224,953, and PCT/US99/05291, all by The General Hospital Corporation, which show that this hypothesis, and related hypotheses regarding the role of reactive oxygen species, has enabled the identification of a number of candidate therapeutic agents. In addition, International patent application No. PCT/US94/11895 by The General Hospital Corporation discloses a diagnostic assay for Alzheimer's disease which is based on the same hypothesis.

Agents which interact with the APP and limit the production of the Aβ peptide are therefore particularly desirable. In vitro studies have shown that the interaction between Cu and APP can cause APP fragmentation and decreased APP processing into Aβ. The binding of divalent copper leads to the oxidation of two cysteine residues of APP, and in turn to formation of an additional disulphide bond (Multhaup et al., 1998; Borchardt et al., 1999). An understanding of the molecular interaction between Cu and the APP is required for the rational development of agonists capable of limiting the production of the Aβ peptide.

Such agonists may have the additional potential to protect cells from damage by blocking Cu(II) binding of APP and thus preventing the formation of reactive axygen species produced by Cu(II) and hydrogen peroxide (Borchardt et al., 1999).

International patent application No. PCT/DE00/00693 by Beyreuther et al. (WO00/51632) shows that APP interacts with divalent zinc and copper ions at two specific sites. They also showed that copper stimulates increased release of the products of the α-secretory metabolic pathway of APP, sAPPα and p3, and reduced release of the products of the β-secretory metabolic pathway, p3.5 and Aβ. Beyreuther et al. therefore proposed that compounds which act as copper agonists could reduce or prevent the formation of Aβ, and therefore would be useful for treatment or prevention of Alzheimer's disease. However, although functional methods of identifying compounds which could be effective for this purpose are described, there is no information provided regarding the structure of the copper-binding site, and therefore this application provides no rational basis for selecting candidate compounds for testing in the functional assays.

The present inventors have now determined the three-dimensional solution structure of the copper-binding domain of APP. Knowledge of the structure of this binding domain provides a basis for the rational identification of compounds which bind to APP and interfere with amyloidogenic processing of APP. It is envisaged that compounds which mimic the effect of, promote or stabilise the binding of copper to the APP will decrease the processing of APP into Aβ, and will reduce the associated APP-Cu(I)-mediated generation of residue oxygen intermediates and will therefore be useful as therapeutic agents.

### SUMMARY OF THE INVENTION

In a first aspect the invention provides a method of identifying a compound which is capable of acting as an agonist of binding of divalent copper ions to APP, comprising the step of identifying a compound which has a conformation and polarity such that it interacts with an amino acid residue in APP selected from the group consisting of:

a) His147, His150, Tyr168 and Met170;

b) Leu136, Gln138, Glu139, Arg140, Met141, Val143, Glu145, His149, Trp150, Val153, Ala154, Thr157, Arg180, ly181, Val182 and Phe184; and

c) His147, His151, Tyr168, Met170, Ala154, Trp150, Lys155, Leu165, Gly169, Phe179, Val182, Glu183 and Phe184.

The regions encompassing the groups of amino acid residues listed here as a), b) and c) are referred to herein as the first, second and third copper-binding sites respectively. Collectively these regions form part of the copper-binding domain of APP. Without wishing to be limited by any proposed mechanism, we believe that region (a) is the principal site to which copper atoms bind.

Preferably a compound identified by this step is then subjected to in vitro and/or in vivo testing for ability to act as an agonist of binding of divalent copper to APP, and for the ability to inhibit the production of Aβ from APP.

Preferably the compound has the ability to interact with the copper-binding domain of APP in such a way that the extent of processing of APP to Aβ or to Aβ-containing fragments in the presence of the compound is reduced, compared to that of APP in the absence of the compound.

Preferably the compound binds to at least two, more preferably at least three, and even more preferably four of the amino acids identified above.

Even more preferably the compound has the ability to penetrate the blood-brain barrier.

It will be clearly understood that the term “identifying” encompasses either designing a new compound, or selecting a compound from a group or library of previously known compounds.

The term “agonist” refers to:

a) a compound which has a conformation and polarity such that the compound itself binds to the copper-binding site of APP;

b) a compound which has a conformation and polarity such that the compound binds to the copper-binding domain of APP at a site other than the copper-binding site, and this enhances or stabilises the binding of copper ions to the copper-binding site; or

c) a compound which has a conformation and polarity such that the compound binds to APP at a site other than the copper-binding site, in which the binding has no effect on copper binding but induces an effect the same as or similar to one which is induced by binding of copper to the copper-binding site.

It will be appreciated that a compound may have more than one of these abilities.

Preferably when the compound has the effect of stabilising binding of copper to the copper-binding domain, the compound stabilises the oxidation state of the copper, thus inhibiting production of toxic Cu(I) ions.

The expression “an effect the same as or similar to one which is induced by binding of copper to the copper-binding site” is to be understood to include effects such as reducing the production of Aβ from APP or induction of dimerization of APP. The skilled person will be aware of suitable techniques for determining whether a compound has one or more such effects.

In one preferred embodiment, the compound interacts directly with the copper-binding site, ie. with one or more amino acids selected from the group consisting of His147, His151, Tyr168 and Met170.

It is contemplated that in this embodiment the compound will be a metal complex which can bind to the imidazole moiety of a histidine residue.

Metal ions capable of binding to the imidazole nitrogen(s) of histidine include Mn, Fe, Co, Ni, Cu, Zn, Ru, Pd, Ag, Cd, Pt, Au, Rh and Hg. Complexes of these metals would be expected to be predominantly four-coordinate tetrahedral or distorted tetrahedral/square planar complexes, five-coordinate complexes with either a trigonal bipyramid or square pyramid configuration, or six-coordinate octahedral or distorted octahedral complexes.

In a second aspect the invention provides a computer-assisted method for identifying compounds potentially able to bind to the copper-binding domain of the APP and decrease the processing of the APP into Aβ or Aβ containing fragments, using a programmed computer comprising the steps of:

(a) inputting into the programmed computer data comprising the atomic coordinates of the APP copper-binding domain, as shown in Appendix A, corresponding to the binding site defined by amino acid residues
(i) His147, His151 and Tyr168 and Met170
(ii) Leu136, Gln138, Glu139, Arg140, Met141, Val143, Glu145, His149, Trp150, Val153, Ala154, Thr157, Arg180, Gly181, Val182 and Phe184, or
(iii) His147, His151, Tyr168, Met170, Ala154, Trp150, Lys155, Leu165, Gly169, Phe179, Val182, Glu183 and Phe184

or a subset thereof;

(b) generating, using computer methods, a set of atomic coordinates of a structure that possesses stereochemical complementarity to the atomic coordinates defined in (a) or a subset thereof, thereby generating a criteria data set;

(c) comparing, using the processor, the criteria data set to a computer database of chemical structures;

(d) selecting from the database, using computer methods, chemical structures which are similar to a portion of said criteria data set; and

(e) outputting the selected chemical structures which are similar to a portion of the criteria data set.

Preferably the method further comprises the step of obtaining a compound with a chemical structure selected in steps (d) and (e), and testing the compound for the ability to decrease processing of the APP into Aβ or Aβ-containing fragments.

In a third aspect the invention provides a computer or a software component thereof for producing a three-dimensional representation of a molecule or molecular complex, which comprises a three-dimensional representation of a homologue of the molecule or molecular complex, in which the homologue comprises a domain that has a root mean square deviation from the backbone atoms of the amino acids of not more than 1.5 Å, in which the computer comprises:

(a) a machine-readable data storage medium comprising a data storage material encoded with machine-readable data, wherein the data comprises the structure coordinates, as shown in Appendix A, of:
(i) His147, His151 and Tyr168 and Met170
(ii) Leu136, Gln138, Glu139, Arg140, Met141, Val143, Glu145, His149, Trp150, Val153, Ala154, Thr157, Arg150, Gly181, Val182 and Phe184, or
(iii) His147, His151, Tyr168, Met170, Ala154, Trp150, Lys155, Leu165, Gly169, Phe179, Val182, Glu183 and Phe184;

(b) a working memory for storing instructions for processing the machine-readable data;

(c) a central-processing unit coupled to the working memory and to the machine-readable data storage medium for processing the machine-readable data into the three-dimensional representation; and

(d) a display coupled to the central-processing unit for displaying the three-dimensional representation.

In one class of embodiments, the three-dimensional representation is of a molecule or molecular complex defined by the set of structure coordinates set out in Appendix A, or wherein the three-dimensional representation is of a homologue of the molecule or molecular complex, the homologue having a root mean square deviation from the backbone atoms of the amino acids of not more than 1.5 Å.

An additional aspect of the invention provides a computer or a software component thereof for determining at least a portion of the structure coordinates corresponding to a three-dimensional structure of a molecule or molecular complex, in which the computer comprises:

(a) a machine-readable data storage medium comprising a data storage material encoded with machine-readable data, in which the data comprises at least a portion of the structural coordinates according to Appendix A;

(b) a machine-readable data storage medium comprising a data storage material encoded with machine-readable data, wherein the data comprise NMR spectral data of the molecule or molecular complex;

(c) a working memory for storing instructions for processing the machine-readable data of (a) and (b);

(d) a central-processing unit coupled to the working memory and to the machine-readable data storage medium of (a) and (b) for performing a transformation of the machine readable data of (a) and for processing the machine-readable data of (b) into structure coordinates; and

(e) a display coupled to the central-processing unit for displaying the structure coordinates of the molecule or molecular complex.

In a fourth aspect the invention provides a compound able to act as an agonist of the binding of copper to the copper-binding domain of APP, wherein the compound is identified by a method according to the invention.

It will be clearly understood that pharmaceutically acceptable salts, derivatives and esters of these compounds are also within the scope of the invention.

In a fifth aspect the invention provides a composition comprising a compound according to the invention, together with a pharmaceutically-acceptable carrier. It will be appreciated that the composition may comprise two or more compounds according to the invention.

In a sixth aspect the invention provides a method of reducing the processing of the APP into Aβ or Aβ-containing fragments, comprising the step of exposing the APP to a compound which acts as an agonist for the binding of divalent copper to the copper-binding domain of APP.

In a seventh aspect the invention provides a method of reducing the amyloidogenic processing of APP, comprising the steps of exposing APP to a compound which acts as an agonist of the binding of divalent copper to the copper-binding domain of APP.

In an eighth aspect the invention provides a method of treating Alzheimer's disease or other amyloid-related condition, the method comprising administering to a subject in need thereof a composition according to the invention.

In one preferred embodiment of the invention, the compound is conjugated to a targeting moiety.

The term “targeting moiety” as used herein refers to a functional group which is covalently linked to a targeting moiety which will specifically bind to or associate with the APP. Suitable targeting moieties include, but are not limited to, polypeptides, nucleic acids, carbohydrates, lipids, APP ligands, antibodies and the like. In a preferred embodiment the targeting moiety has a hydrophobic region which interacts with the APP. For example, the targeting moiety may include a fatty acid molecule.

In a particularly preferred embodiment, the ligand-targeting moiety complex is able to pass through the blood-brain barrier.

Preferably in each of the fourth to eighth aspects the compound has a conformation and polarity such that it binds to at least one, preferably at least two, more preferably at least three, and most preferably four amino acid residues in APP selected from the group consisting of:

a) His147, His151 and Tyr168 and Met70;

b) Leu136, Gln138, Glu139, Arg140, Met141, Val143, Glu145, His149, Trp150, Val153, Ala154, Thr157, Arg180, Gly181, Val182 and Phe184; and

c) His147, His151, Tyr168, Met170, Ala154, Trp150, Lys155, Leu165, Gly169, Phe179, Val182, Glu183 and Phe184.

It is preferred that the compound has a high affinity for the selected target site. For in silico screening using computer modelling systems, the affinity constant is preferably ≦1 mM, more preferably ≦1 nM. For in vitro binding as assessed by NMR, the affinity constant is preferably ≦1 nM.

The compounds of the invention may be formulated into pharmaceutical compositions, and administered in therapeutically effective doses. By “therapeutically effective dose” is meant a dose which results in the inhibition of the processing of APP into Aβ or Aβ-containing fragments. The appropriate dose will be ascertainable by one skilled in the art using known techniques.

The pharmaceutical compositions may be administered in a number of ways, including, but not limited to, orally, subcutaneously, intravenously, intraperitoneally and intranasally.

The dosage to be used will depend on the nature and severity of the condition to be treated, and will be at the discretion of the attending physician or veterinarian. The most suitable dosage for a specific condition can be determined using normal clinical trial procedures.

While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as felids, canids, bovids, and ungulates.

Methods and pharmaceutical carriers for preparation of pharmaceutical compositions are well known in the art, as set out in textbooks such as Remington's Pharmaceutical Sciences, 19th Edition, Mack Publishing Company, Easton, Pa., USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

Amyloid deposition has also been implicated in the pathogenesis of the neurodegenerative disease, such as Parkinson's disease, amyotrophic lateral sclerosis, and cataract. Thus, while the invention is described in detail in relation to Alzheimer's disease, it is also applicable to amyotrophic lateral sclerosis, motoneuron disease, cataract, Parkinson's disease, Creutzfeldt-Jacob disease, Hungington's disease, dementia with Lewy body formation, multiple system atrophy, Hallerboden-Spatz disease, and diffuse Lewy body disease, or cataracts.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE FIGURES

FIG. 1 is a schematic representation of APP, showing important regions of the molecule. The N-terminal growth factor domain is followed by the copper-binding domain (CuBD), an acidic region, Kunitz-type protease inhibitor and Ox-2 domains which occur in some APP isoforms, a series of domains known to bind carbohydrate, a transmembrane (TM) region, and a cytoplasmic tail. The location of the Aβ region, a major component of Alzheimer's disease plaques, is also shown. The sequence of the CuBD is shown, with the copper-binding amino acid residues in large bold type, and the cysteine residues which are involved in disulphide bridges shown in underlined bold type.

FIG. 2 shows the solution structure of APP124-189.

a. Stereoview of the backbone (N, C^{α}, C) traces of the 21 lowest energy structures for APP124-189 superimposed over backbone atoms (N, C^{α}, C) of residues 128-189.

b. A ribbon depiction of the NMR structure closest to the geometric average of APP124-189.

C. Surface characterization of APP124-189. Electrostatic molecular surface rendering of the NMR structure closest to the geometric average of APP124-189. Surfaces are shaded to indicate electrostatic charge, with regions with electrostatic potential <−8 k_{B}T light grey and those >+8 k_{B}T dark grey (k_{B}, Boltzmann constant, T, absolute temperature).

FIG. 3 shows NMR spectra of APP124-189, illustrating the effects of addition of metal ions.

a. The top spectrum is a 600 MHz ¹H spectrum of unlabelled APP124-189; the bottom spectrum is after the addition of Zn²⁺. Peaks due to the S-methyl Hε of Met170, Hδ2 of His150 and the Hδ1 and Hε1 of Tyr168 that are visible in the top spectrum disappear from the bottom spectrum upon the addition of Zn²⁺ have been labelled.

b. [¹H, ¹⁵N] HSQC spectra of ¹⁵N-labeled APP124-189 before (spectrum on the left) and after (spectrum on the right) the addition of one equivalent of Cu²⁺.

C. Changes in the [¹H, 15N] HSQC spectrum upon addition of Cu²⁺ are mapped on to the surface of APP124-189, unaffected residues are shown in dark grey, while those residues whose backbone amides were perturbed by the presence of Cu²⁺ are shown in light grey.

d. A region of the [¹H, ¹³C] HSQC spectra of ¹⁵N, ¹³C-labeled APP124-189 where the S-methyl Hε and Cε resonances of the methionine residues are visible. The top spectrum shows the S-methyl Hε and Cε resonances of 141 on the left and 170 on the right. The middle spectrum shows that the effect of adding one equivalent of Cu²⁺ is the disappearance of the peak due to Met170. The bottom spectrum shows that a new peak is visible in the region of the [¹H, ¹³C] spectrum associated with the S-methyl of methionine, after APP124-189 has incubated with Cu²⁺ for 48 hours.

FIG. 4 shows orthogonal views of the putative metal binding site, residues His147, His151, Tyr168 and Met170. These views show that the orientation of the residues is such that the configuration around any metal bound in this site is tetrahedral, and that the binding site is a surface exposed site.

FIG. 5 shows a model of a metal ion in a tetrahedral configuration bound to His147, His150, Tyr168 and Met170 of APP124-189.

FIG. 6 shows docking of (2,5-dimethyl-phenyl)-carbamic acid napthalen-2-yl ester (Compound 42613) to the principal copper-binding site of APP, encompassing residues His147, His151, Tyr168 and Met170, as demonstrated in an in silico modelling system.

FIG. 7 shows docking of 3-(4-Iodo-phenyl)-1-phenyl-imidazo [1-5-a]pyridine (Compound 21056) to the secondary copper-binding site of APP which is located at the back the copper-binding domain and encompasses the residues listed in (b) above, as demonstrated in an in silico modelling system.

### DETAILED DESCRIPTION OF THE INVENTION

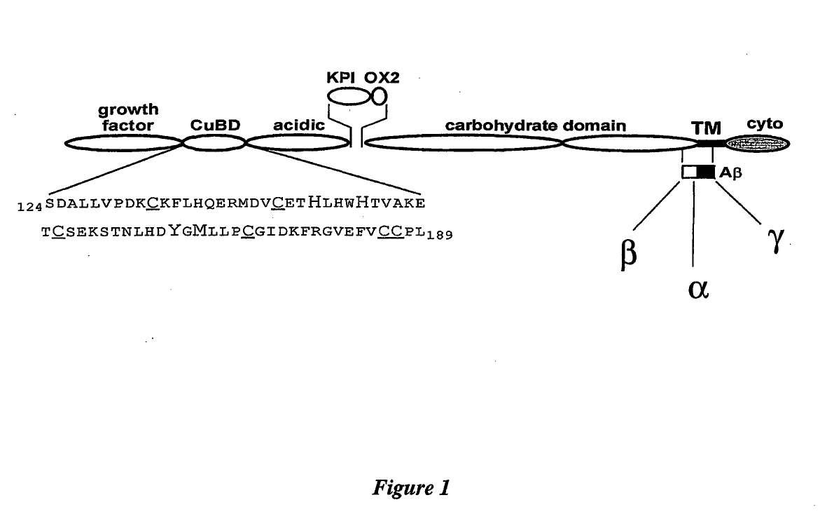

The general structure of APP is illustrated schematically in FIG. 1, which shows the important regions of the molecule. It can be seen that the copper-binding domain is near the N-terminal of the molecule, whereas the region which gives rise to Aβ is close to the C-terminal. The sequence APP124-189 from the copper-binding domain was selected by analysis of the crystal structure of the growth factor domain and sequencing of the C-terminal region.

The derivation of the CuBD construct, APP124-189, was based upon our previous work, in which we solved the structure of APP28-123 (Rossjohn et al., 1999). Those studies defined the C-terminal end of the first APP domain, also called the growth factor domain, as residue 123. Therefore the start of the second domain, CuBD, commenced at residue 124. The C-terminal end of the CuBD was chosen as residue 189. This corresponded to the end of the putative zinc binding site (Bush et al., 1993).

APP133-189 corresponds to a truncated version of the CuBD. Based on our copper-binding studies using NMR, the free N-terminus was involved in artefactual copper-binding. This free N-terminus is not present in physiologically expressed APP. To remove the influence of this artefactual Cu binding site, APP133-189 was generated. Furthermore, residues 124-132 were predominantly unstructured and this may have prevented crystal formation; in fact we have found that APP133-189 readily forms crystals as a result of removal of these residues. The APP133-189 will be valuable for future crystallisation structure studies.

Previous studies by Multhaup and colleagues proposed that the reduction of Cu(II) by the CuBD results in the formation of an intramolecular disulphide bond between cysteines 144-158 (Multhaup et al., 1998). The results presented herein indicate an alternative mechanism, whereby the reduction of Cu(II) to Cu(I) does not require the formation of an intramolecular disulphide bond between cysteines 144-158. We have found that although the APP124-189 protein has no free cysteines, it is able to reduce Cu(II) to Cu(I). Our structure proposes that methionine is a suitable substrate to be oxidised in order for Cu(II) to be reduced. The NMR structure reported here for the first time has properly defined the copper-binding site beyond the histidine residues at 147, 149 and 151 proposed by Multhaup and colleagues (see below).

In vitro studies have shown that the interaction between Cu and APP can cause APP fragmentation and decreased APP processing into Aβ. In order to define the interaction between copper and the APP at the molecular level, the inventors have solved the NMR structure of the APP copper-binding domain (APP residues 124-189). The co-ordinates for this structure are provided in Appendix A.

The primary observation was that the binding of copper to the copper-binding site of APP, which leads to processing of APP to Aβ, occurs at a site near the surface of the molecule. Modulation of this binding could occur via three possible mechanisms. Firstly, an agonist molecule could exert its action via direct binding to the copper-binding site; alternatively, the agonist could bind to a “secondary shell”, which in turn perturbs the copper-binding domain. A third alternative is that the agonist binds to a separate site more distantly located on the APP molecule.

The three-dimensional structure of APP124-189 has three disulfide bonds, and consists of an α-helix overlying a triple-stranded β-sheet. The surface is highly charged, with several areas of high negative and positive potential. On the surface of the molecule there are two basic patches but no extensive hydrophobic area. The metal-binding properties of APP124-189 to Zn(Gly)₂²⁺, Ni(Gly)₂²⁺ and Cu(Gly)₂²⁺ showed resonance changes in His147, His151 and Tyr168. The side-chains of these residues, together with Met170, are orientated so that they form a favourable metal binding site. Very small movements in their side-chains are required for metal binding to occur in a tetrahedral fashion. No previous examples of coordination spheres incorporating both methionine and tyrosine could be identified in the literature, indicating that the APP CuBD utilizes a novel Cu-binding site.

This information provides a rational basis for the development of compounds which mimic Cu²⁺, ie. which inhibit processing of the APP into Aβ or Aβ-containing fragments. Accordingly, these compounds are likely to have therapeutic value in the treatment of diseases such as Alzheimer's disease.

The general principles of drug design, which are discussed below, can be applied by persons skilled in the art to produce compounds which preferably bind to the copper-binding site defined by the His147, His151 and Tyr168 and Met170 residues of the APP and inhibit the processing of APP into the Aβ peptide.

The inventors have also identified two additional regions on the copper-binding domain of the APP, which are thought to be involved in interactions which enhance or stabilise the binding of copper to the copper-binding site.

The first of these regions, region (b), is located on the back of the copper-binding domain of APP ie. on the opposite side to the copper-binding site, and encompasses residues Leu136, Gln138, Glu139, Arg140, Met141, Val143, Glu145, His149, Trp150, Val153, Ala154, Thr157, Arg180, Gly181, Val182 and Phe184.

The second of these regions, region (c), is located on the same side of APP as the copper-binding site, and encompasses residues His147, His151, Tyr168, Met170, Ala154, Trp150, Lys155, Leu165, Gly169, Phe179, Val182, Glu183 and Phe184.

It is envisaged that the binding of ligands to these regions may enhance, stabilise or mimic the binding of copper to the copper-binding site of APP, thereby reducing amyloidogenic processing of APP.

The preferential binding of ligands to the copper-binding site or other sites on the copper-binding domain of APP, preferably with an affinity in the order of 10⁻⁸M or better, may arise from enhanced stereochemical complementarity relative to naturally-occurring ligands.

Pursuant to the present invention, such stereochemical complementarity is characteristic of a molecule which matches intra-site surface residues lining the groove of the first copper-binding site (eg. His147, His151 and Tyr168 and Met170) or other binding region identified herein. By “match” we mean that the identified portions interact with the surface residues, for example, via hydrogen bonding or by entropy-reducing van der Waals interactions which promote desolvation of the biologically active compound within the site, in such a way that retention of the biologically active compound within the groove is energetically favoured.

It will be appreciated that it is not necessary that the complementarity between ligands and the copper-binding site extend over all residues lining the groove in order to stabilise binding of the natural ligand. Accordingly, ligands which bind to some, but not all, of the residues lining the groove are encompassed by the present invention.

In general, the design of a molecule possessing stereochemical complementarity can be accomplished by means of techniques which optimize, either chemically or geometrically, the “fit” between a molecule and a target receptor. Suitable such techniques are known in the art. (See Sheridan and Venkataraghavan, 1987; Goodford 1984; Beddell 1985; Hol, 1986; and Verlinde 1994, the respective contents of which are hereby incorporated by reference. See also Blundell 1987).

Thus there are two preferred approaches to designing a molecule according to the present invention, which complements the shape of the copper-binding site. In the first of these, the geometric approach, the number of internal degrees of freedom, and the corresponding local minima in the molecular conformation space, is reduced by considering only the geometric (hard-sphere) interactions of two rigid bodies, where one body (the active site) contains “pockets” or “grooves” which form binding sites for the second body (the complementing molecule, as ligand). The second approach entails an assessment of the interaction of different chemical groups (“probes”) with the active site at sample positions within and around the site, resulting in an array of energy values from which three-dimensional contour surfaces at selected energy levels can be generated.

The geometric approach is illustrated by Kuntz et al. (1982), the contents of which are hereby incorporated by reference, whose algorithm for ligand design is implemented in a commercial software package distributed by the Regents of the University of California and further described in a document, provided by the distributor, entitled “Overview of the DOCK Package, Version 1.0,”, the contents of which are hereby incorporated by reference. Pursuant to the Kuntz algorithm, the shape of the cavity represented by the copper-binding site is defined as a series of overlapping spheres of different radii. One or more extant databases of crystallographic data, such as the Cambridge Structural Database System maintained by Cambridge University (University Chemical Laboratory, Lensfield Road, Cambridge CB2 1EW, U.K.) and the Protein Data Bank maintained by Brookhaven National Laboratory (Chemistry Dept. Upton, N.Y. 11973, U.S.A.), is then searched for molecules which approximate the shape thus defined.

Molecules identified in this way, on the basis of geometric parameters, can then be modified to satisfy criteria associated with chemical complementarity, such as hydrogen bonding, ionic interactions and van der Waals interactions.

The chemical-probe approach to ligand design is described, for example, by Goodford (1985), the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the copper-binding site with different chemical probes, e.g., water, a methyl group, an amine nitrogen, a carboxyl oxygen, and a hydroxyl. Favoured sites for interaction between the active site and each probe are thus determined, and from the resulting three-dimensional pattern of such sites a putative complementary molecule can be generated.

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3 DB Unity (Tripos Associates, St. Louis, Mo.).

Programs suitable for pharmacophore selection and design include: DISCO (Abbott Laboratories, Abbott Park, Ill.), Catalyst (Bio-CAD Corp., Mountain View, Calif.), and ChemDBS-3D (Chemical Design Ltd., Oxford, U.K.).

Databases of chemical structures are available from a number of sources including Cambridge Crystallographic Data Centre (Cambridge, U.K.) and Chemical Abstracts Service (Columbus, Ohio).

De novo design programs include Ludi (Biosym Technologies Inc., San Diego, Calif.), Sybyl (Tripos Associates) and Aladdin (Daylight Chemical Information Systems, Irvine, Calif.).

Those skilled in the art will recognize that the design of a mimetic compound may require slight structural alteration or adjustment of a chemical structure designed or identified using the methods of the invention.

This aspect of the invention may be implemented in hardware or software, or a combination of both. However, the invention is preferably implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

It is contemplated, without wishing to limit the invention, that a suitable compound may be a metal complex that can exchange or bind functional moieties such as histidine. Preferably the metal complex is capable of binding between 1 and 4, more preferably 3 or 4 of the amino acid residues His147, His151, Tyr168 and Met170 of the copper-binding domain of amyloid precursor protein. Metal ions capable of binding to the imidazole nitrogen(s) of histidine include Mn, Fe, Co, Ni, Cu, Zn, Ru, Pd, Ag, Cd, Pt, Au, Rh and Hg. Complexes of these metals would be expectecd to be predominantly four coordinate tetrahedral (distorted tetrahedral)/square planar complexes, five coordinate complexes with either a trigonal bipyramid or square pyramid configuration or six coordinate octahedral (or distorted octahedral) complexes.

Compounds identified by the methods of the present invention may be assessed by a number of in vitro and in vivo assays. For example, the identification of ligands which bind to the copper-binding site may be undertaken using a solid-phase receptor binding assay. Ligands can be screened in a cell-based assay by measuring their effect on APP processing and Aβ production. Binding affinity for candidate ligands may be measured using biosensor technology.

Assays to determine the binding of metal complexes to APP may be performed by NMR and UV-Visible spectroscopy and ESR for paramagnetic metals. Assays are available for measuring Cu/Fe reduction, hydrogen peroxide generation, hydroxyl radical generation, and carbonyl generation, all of which assess the redox capacity of APP in the presence of Cu and Fe.

The invention is further described below in detail with reference to the following, non-limiting examples.

### EXAMPLE 1

### Expression and Purification of Recombinant APP124-189 and APP133-189

The sequence encoding APP124-189 (SEQ ID NO:1) was amplified by PCR using the primers

GCT CGA GAA AA GAG AGG CTA GTG ATG CCC TTC TCG

(primer 1; SEQ ID NO:2) and

GAA TTC TTA CAG TGG GCA ACA CAC AAA CTC

(primer 2; SEQ ID NO:3).

The PCR product was cloned as a XhoI-EcoRI fragment into the Pichia pastoris vector pIC9 (Invitrogen) and then transformed into P. pastoris strain GS115 as previously described (Henry et al., 1997). Expressing clones were identified by analysing the culture supernatants by silver stain-SDS-PAGE.

APP 124-189, whose structure is shown schematically in FIG. 1, was expressed as a secreted protein in the yeast Pichia pastoris and purified by a two-step purification scheme to homogeneity, as judged by mass spectroscopy and N-terminal sequencing.

Isotopically-labelled protein was prepared by the protocol of Laroche et al (Laroche et al., 1994). ¹⁵N-single labelled APP124-189 was produced using FM22 basic medium, which contains 5 g ¹⁵NH₄Cl as the sole nitrogen source. A five ml overnight culture was inoculated into 400 ml growth medium (FM22 basic, 0.5%-glucose, 0.1% PTM1 salts, 0.06M KOH). After 48 h the cells were collected by centrifugation and resuspended into induction medium (FM22 basic medium, 0.5% methanol, 0.1% PTM1 salts) and shaken for 48 hr. The ¹³C, ¹⁵N-double labelled APP124-189 was expressed as described above, except that the induction medium was FM22 basic medium, 0.5% ¹³C-methanol, 0.1% PTM1 salts. After 24 hr another 0.5% ¹³C-methanol was added, and then the culture was shaken for a further 24 hr. The cells were pelleted by centrifugation and the supernatant kept for protein purification.

APP124-189 was purified to homogeneity in two steps. The supernatant was concentrated, and the buffer was exchanged into 20 mM TRIS buffer pH 8.5 containing 5 mM EDTA, and then applied to a QHyperD 1.6×13 cm column (Biosepra, France), equilibrated in the same buffer. The APP124-189 protein was eluted in the column flow-through, then concentrated and then applied to a Superdex 75 HR 10/30 gel filtration column (Amersham-Pharmacia, Australia) in 20 mM sodium phosphate buffer pH 6.8 containing 1 mM EDTA. The APP124-189 eluted as a single peak with a molecular weight of ˜11 kDa. N-terminal amino acid sequencing and mass spectrometery (MALDI-TOF) analysis confirmed that the N-terminus was intact, and that the mass correlated to the predicted sequence. The APP124-189 protein was concentrated by ultrafiltration to a final concentration of 5 mg/ml in 20 mM phosphate pH 6.8 buffer.

APP133-189 was expressed and purified in the same way as for APP129-189.

### EXAMPLE 2

### NMR Spectroscopy and Spectral Assignments

NMR spectra were acquired at 30° C. using a Bruker DRX-600 spectrometer equipped with triple-resonance pulsed-field gradient probes. Sequential resonance assignments were made using a series of triple resonance spectra (Sattler et al., 1999) acquired on either uniformly ¹⁵N— or ¹³C, ¹⁵N-labelled APP124-189 using the methods described by Day et al. (1999). Spectra were obtained on samples which typically contained 0.5 mM protein in 50 mM phosphate buffer (pH 6.9) at 30° C., and 1 mM EDTA, which was either removed or titrated out in the metal-binding studies. APP124-189 had good solution properties at pH 6.9, and was stable for months in the presence of EDTA at 0.5 mM.

Inductively-coupled plasma mass spectrometry analysis revealed that APP124-189 had very low levels of bound metal, suggesting that it was in the apo form. An essentially complete set of resonance assignments was determined from spectra acquired using ¹⁵N and ¹³C, ¹⁵N-labelled protein. ¹⁵N resonances were not observed for Asp125 and Ile176. Ten hydrogen bond restraints were also employed along the helix. There are six cysteine residues within this domain, and these form three disulfide bonds. An Ellman's test confirmed that there were no free thiols present, and chemical methods had previously determined the disulfide pairings to be 133-186/187, 158-186/187 and 144-174. Klaus et al. (1993) have shown that Cᵢ^{α}H/Cⱼ^{β}H and Cᵢ^{β}H/Cⱼ^{β}H NOEs have a positive predictive value for a disulfide pairing between half-cystines i and j.

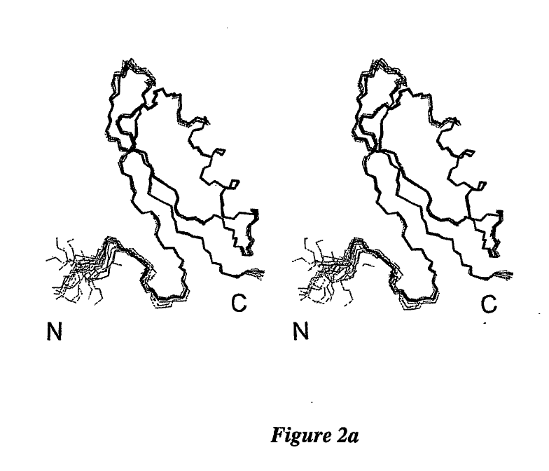

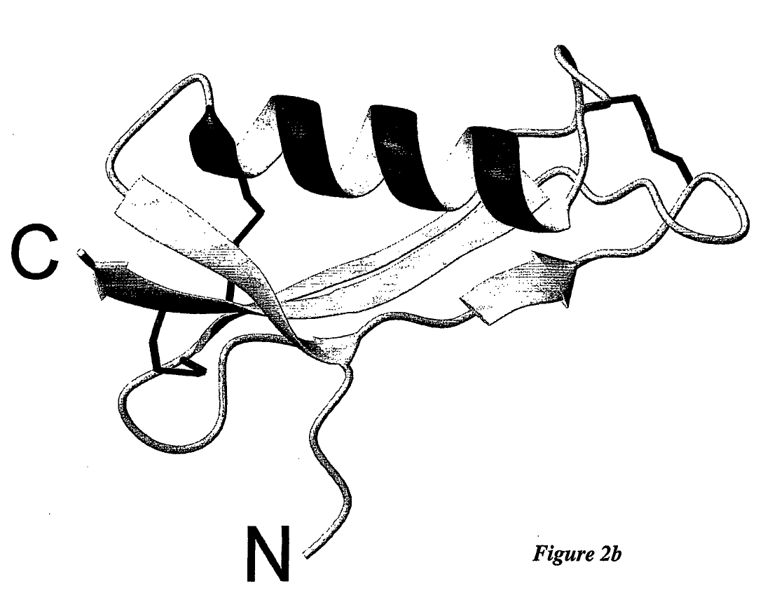

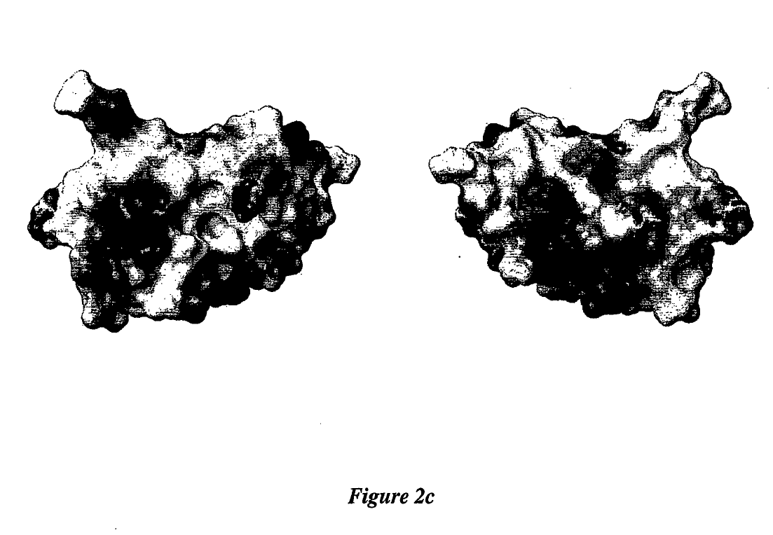

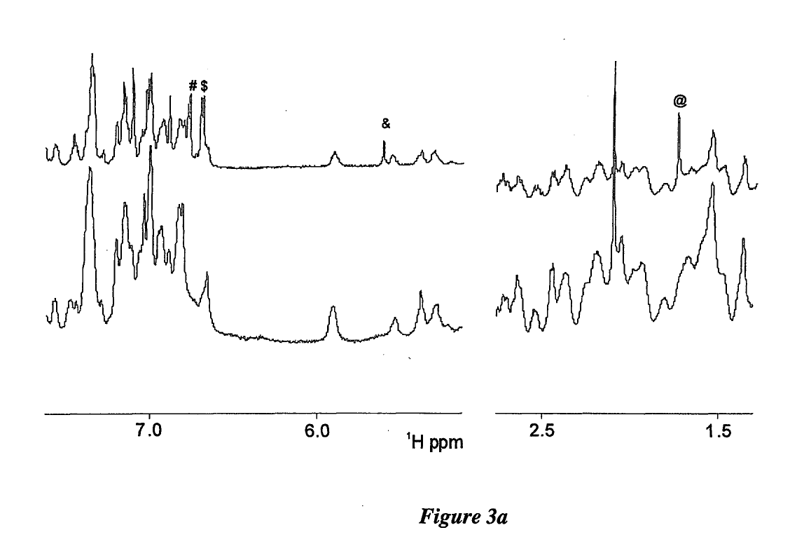

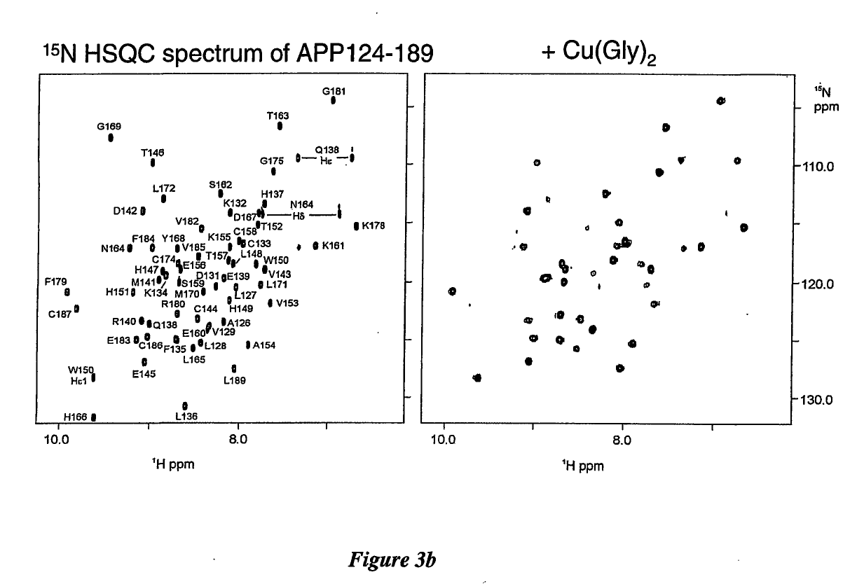

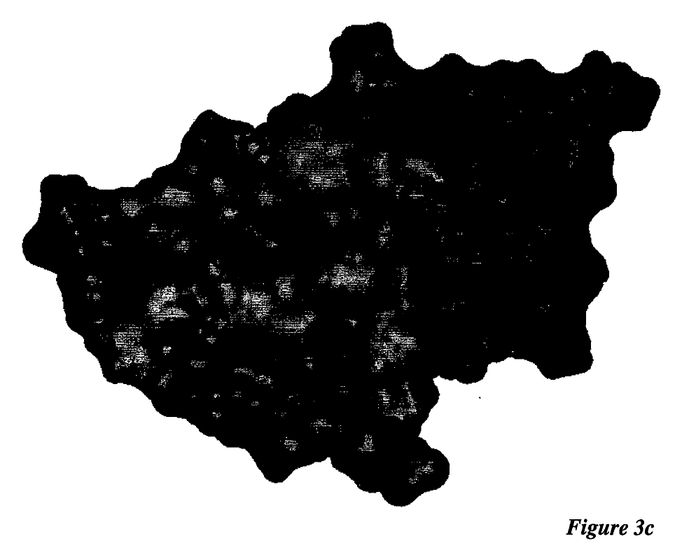

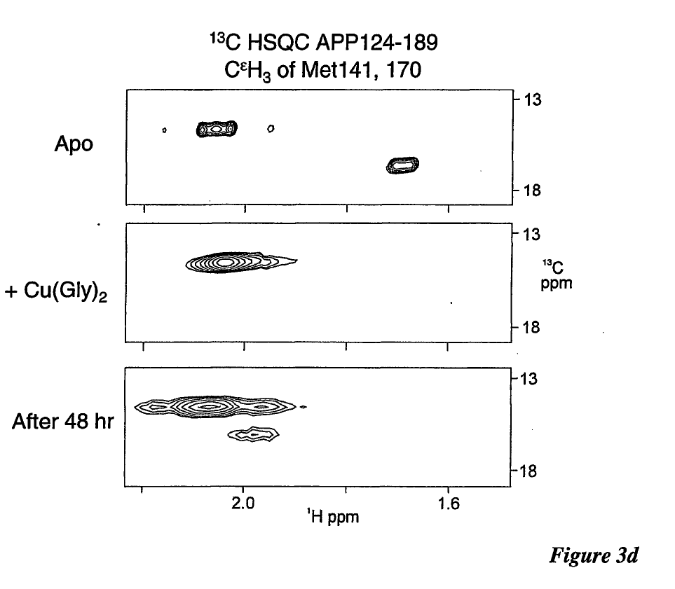

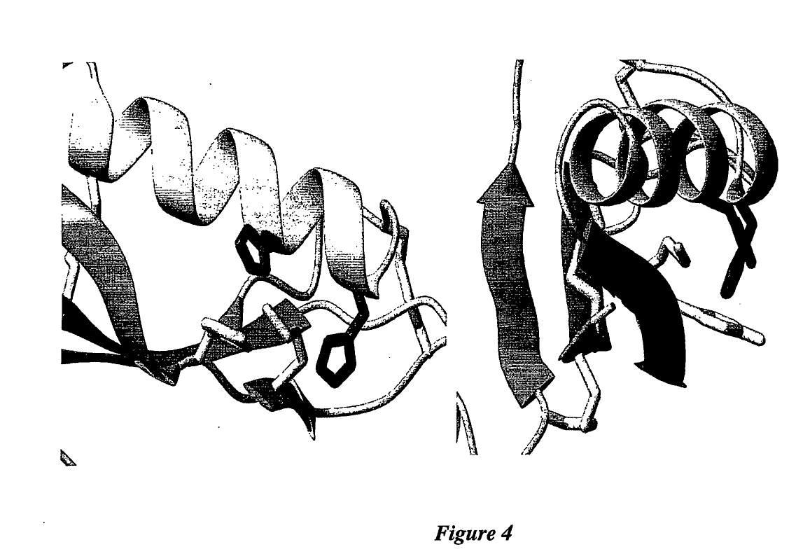

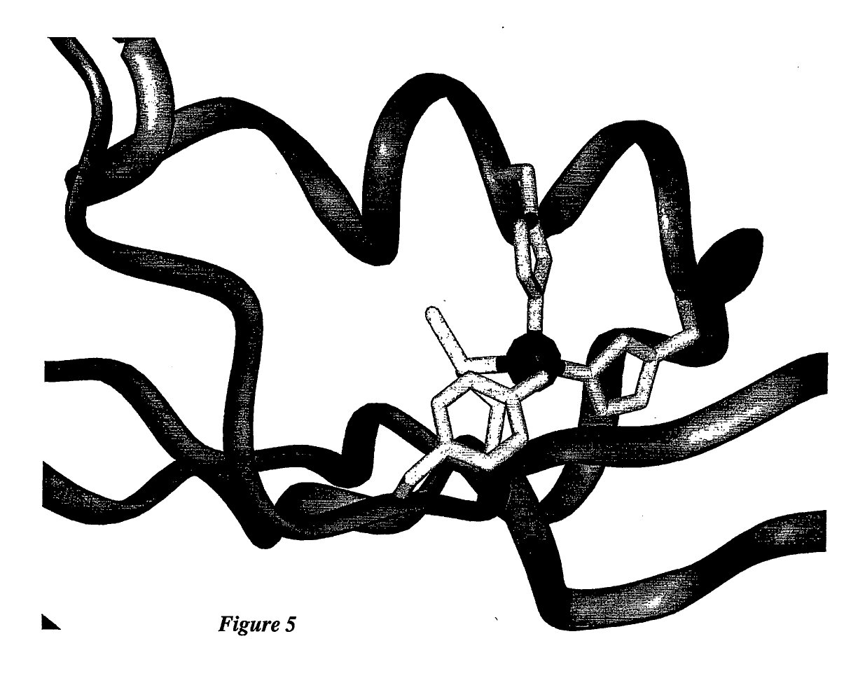

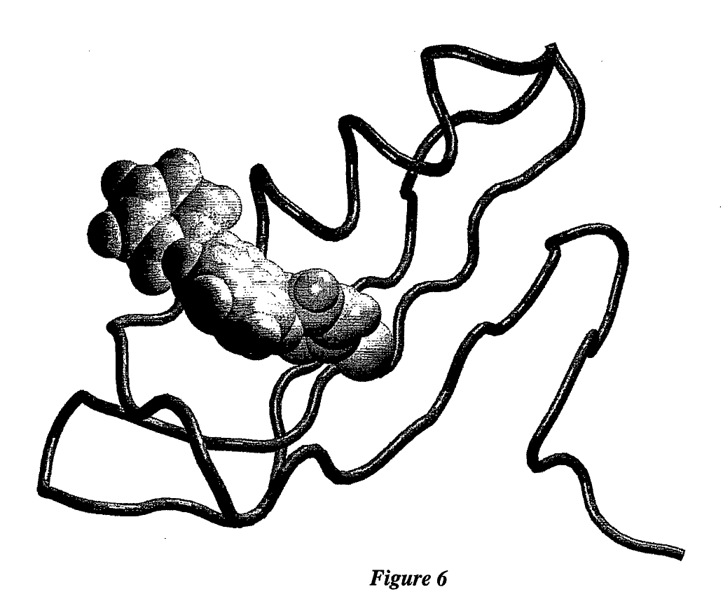

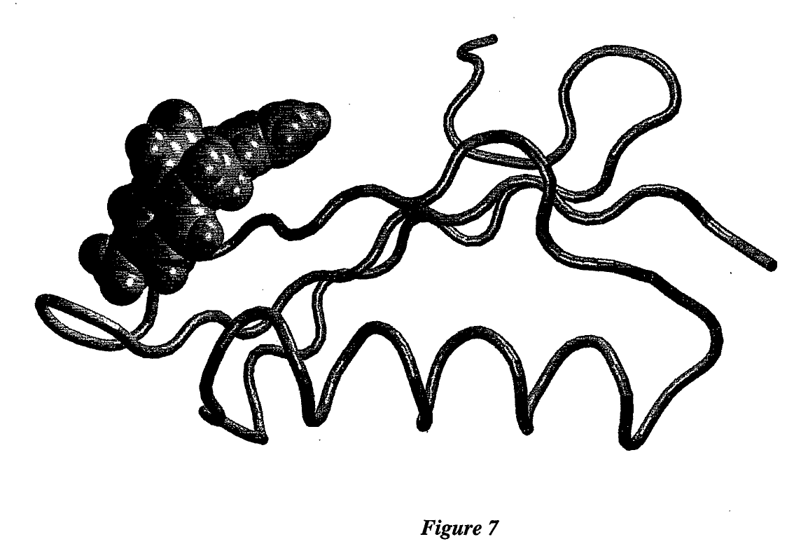

Cᵢ^{α}H/Cⱼ^{β}H and Cᵢ^{β}H/Cⱼ^{β}H NOEs were observed between residues 133 and 187 as well as between 158 and 186, and as a result of this the structure of APP124-189 was determined with the 133-187, 158-186 and 144-174 disulfide pairings (1-6, 2-4). The structures were calculated using the methods reported by Day et al. (1999). The final 21 structures, depicted in FIG. 2a, were selected on the basis of their stereochemical energies, and structural statistics are presented in Table 1.

TABLE 1
Structural Statistics for the 21 Energy-Minimised
Structures of APP124-189 from CNS.^{a}

| | |
|---|---|
| Total distance constraints | 1233 |
| Sequential (\|i − j\| = 1) | 321 |
| Short-range (1 < \|i − j\| < 5) | 229 |
| Long-range (\|i − j\| ≧ 5) | 532 |
| Intra-residue | 151 |
| Hydrogen bonds^{a} | 10 |
| Total angle constraints | 111 |
| φ | 59 |
| ψ | 21 |
| χ1 | 31 |
| RMS deviations from experimental distance | 0.0216 ± 0.0009 |
| restraints Å (1241)^{b} | |
| RMS deviations from experimental dihedral | 0.83 ± 0.05 |
| restraints (deg) (109)^{b} | |
| RMS deviations from idealised geometry | |
| bonds (Å) | 0.004 ± 0.00009 |
| angles (deg) | 0.50 ± 0.02 |
| impropers (deg) | 0.41 ± 0.02 |
| Mean pairwise RMSD (Å) | |

| | Backbone heavy atoms | All heavy atoms |
|---|---|---|
| Residues 127-189 | 0.43 ± 0.16 | 1.13 ± 0.13 |
| Residues 133-139, 147-159, | 0.17 ± 0.05 | 0.78 ± 0.11 |
| 162-167, 181-188 | | |

^{a}The best 21 structures after energy minimisation using the program CNS (Brunger et al., 1998).
^{b}The numbers of restraints are shown in parentheses. None of the structures had distance violations > 0.2 Å or dihedral angle violations > 5°.

The structures are well ordered, with only the three N-terminal residues unstructured (angular order parameter <0.9), and they have good stereochemical properties, with 15 over 98% of backbone angles falling in the allowed regions of the Ramachandran plot. {¹H} ¹⁵N heteronuclear NOE data (data not shown) indicate that, with the exception of residues near the N-terminus, the molecule is rigid along the entire sequence. The pairwise root mean square deviation (RMSD) for the ordered residues (127-189) is 0.43 ±0.16 over the backbone atoms (N, C^{α}, C), and 0.17±0.05 for the residues involved in secondary structure elements.

The three-dimensional structure of APP124-189, shown in FIG. 1b, consists of an α-helix (residues 147-159) overlying a triple stranded β-sheet (residues 133-139, 162-167 and 181-188). Residues 133-139 constitute the first strand of the β-sheet, residues 181-189 the second strand, and residues 162-167 the third strand. The disulfide bond between Cys133-Cys187 links the first two strands of the β-sheet, while the 158-186 bond links the α-helix to the middle strand of the P-sheet. The 144-174 disulfide bond connects two loops: Cys144 is in the loop between the first strand of the β-sheet and the α-helix, while Cys174 is in the loop which connects the second and third strands of the β-sheet.

There are very few buried hydrophobic residues in the vicinity of this disulfide bond, which is therefore probably very important in stabilising the structure in a region which does not have any secondary structure. In addition to the three disulfide bonds there is a small hydrophobic core which assists in stabilising the structure; this core consists of small segments of residues from each of the secondary structure elements, Leu136, Trp150, Val153, Ala154, Leu165, Met170, Val182 and Val185. Leu172 has a positive φ angle, as determined by the presence of a large intraresidue NH to C^{α}H NOE and weak (i+1) NOE (Ludvigsen & Poulsen. (1992).

As illustrated in FIG. 1c, the surface of APP124-189 is highly charged, with several areas of high negative and positive potential. There is a basic patch on each of the two faces of the protein. One patch consists of residues Lys132, Lys134, Lys161, and on the opposite side of the molecule one face of the helix, His147, His151 and Lys155, also gives rise to a basic patch. Acidic regions Glu156, Glu160 on one face and Glu183, Asp167, Asp131 on the opposite are smaller. There is no extensive hydrophobic area on the surface of APP124-189.

Mass spectrometry revealed that the purified protein was essentially free of metal ions, confirming that it was in the apo form. Titration of Cu(II) into a solution of CuBD resulted in the broadening of some resonances in the NMR spectrum, as would be expected for resonances close to a paramagnetic centre such as Cu(II). Analysis of the pattern of peaks in the 2D [¹H, ¹⁵N] HSQC spectrum that were broadened by Cu(II), as shown in FIG. 3a, suggested the presence of two binding sites, one centred on His147, His151, Tyr168 and Met170 and the other involving the N-terminus. The copper-binding site at the N-terminus is not physiologically relevant, since in the intact protein the N-terminus is connected to the growth factor domain (see FIG. 1). There was a general decrease in the signal-to-noise ratio of the spectrum following Cu(II) addition, suggesting that higher order aggregates were being formed. To further characterize the metal binding site, the diamagnetic ions Zn(II) and Ni(II) were each titrated into CuBD solutions; similar changes in the NMR spectra were observed with either metal. Decreases in signal-to-noise consistent with metal ion-induced aggregation were observed with Zn(II) addition, leading to a visible precipitate. Resonances corresponding to the aromatic protons of Tyr168 broadened and disappeared, as did C^{ε}H₃ protons of Met170 (FIG. 3b). The backbone amide resonances from His147, His151 and Tyr168 in the 2D [¹H, ¹⁵N] HSQC spectrum broadened beyond detection on addition of excess metal ion.

The broadening of resonances upon addition of metal ions is evidence for chemical exchange between the metal bound and apo forms of the protein at an intermediate rate, which implies a K_{d} in the micromolar range. The lack of change in resonances distant from the immediate metal binding sites indicated that there was no significant structural alteration upon metal binding. To demonstrate a role for Met170 in the binding and reduction of Cu(II), changes in the 2D [¹H, ¹³C] HSQC spectrum of CuBD upon addition of Cu(II) were monitored, with the ¹H and ¹³C chemical shifts of the C^{ε}H₃ and C^{ε} of methionine being diagnostic. Initially, the S-methyl resonance of Met170 broadened beyond detection, whereas the resonance of Met141, the only other methionine in CuBD, was unaffected. After incubating the sample at 30° C. for 48 hr a new resonance was observed, with ¹H and ¹³C chemical shifts typical of C^{ε}H₃ and C^{ε} from methionine, indicating the presence of a modified Met170, as shown in FIG. 3c.

Examination of the structure around the residues whose NMR chemical shifts were affected by the binding of metals shows that residues His147, H149, Tyr168 and Met170 are clustered together in the three-dimensional structure so that the side-chains of these residues are presented in a way that would be favourable for metal binding. This is illustrated in FIG. 4. The structural orientation of these residues shows that very small movements of the side-chains of these four residues are all that is required before they could bind a metal in a tetrahedral fashion, as shown in FIG. 5.

A search of the protein data base did not find any similar coordination spheres for M²⁺, although peptidylglycine monoxygenase (PDB accession number 1 phm) contains a surface-available redox-active Cu²⁺ binding site which consists of two histidine residues, a methionine residue, and a water molecule in a tetrahedral coordination sphere [Prigge et al. 1997]. This protein is found primarily in the pituitary gland, and its function is to C-terminally amidate bioactive peptides. There were no other structurally similarities between peptidylglycine monoxygenase and APP124-189.

While no examples of coordination spheres incorporating both methionine and tyrosine could be identified in the literature, there are several examples of copper coordination spheres which include the side-chains of tyrosine residues. An example is galactose oxidase (PDB accession number 1 gof); the copper coordination sphere of this protein includes two histidines and two tyrosine residues, and its function is the catalysis of the stereospecific oxidation of a broad range of primary alcohol substrates. The copper coordination site, which is the active site of this enzyme, has 5-coordinate square pyramidal coordination sphere. Acetate is the fifth ligand in the structure, and Tyr495 occupies the apex position of the pyramid (Ito et al. 1991). There no other structural similarities between APP124-189 and galactose oxidase.

The secondary shell about the metal binding site of APP124-189, defined as those residues within 3.5 Å of a residue coordinating the metal (Karlin et al., 1997) is predominantly hydrophobic, with Ala154, Trp150, Leu165, Phe179, Val182 and Phe184 all falling with this region. Two hydrophilic residues, Lys155 and Glu183, are also within 3.5 Å of His151 and Tyr168 and of His47 and Met170, respectively.

The copper-binding site involving His137 and the N-terminus is considered to be an artefact of this particular construct, as the free amine of the N-terminus is a good metal ligand, as indicated by the binding of copper or nickel to albumin. The first two residues of the APP124-189 construct are Ser124 and Asp125; these are exactly the same as the first two residues of thioredoxin. The N-terminus of thioredoxin is a known copper-binding site, and its crystal structure (PDB accession code 2 trx) has a copper which is bound to the N-terminus, a deprotonated backone amide (from D2) and the side-chain carboxyl of the Asp residue, while the fourth ligand is water (Holmgren et al. 1975). This metal-binding site plays no known role in thioredoxin, and is thought to be opportunistic. In the structure of APP124-189 a better fourth ligand than water, His137, is available, as histidine has a high affinity for Cu²⁺. However, this second copper-binding site is an artefact of the construct which was used, since in the intact protein there is no free amino group at position 124.

The binding of Cu²⁺ to the tetrahedrally-arranged His147, His151, Tyr168 and Met170 illustrated in FIG. 4, may explain the redox chemistry associated with copper-binding to APP. In general Cu²⁺ favours a square planar coordination sphere about the metal, while Cu⁺ generally prefers a tetrahedral arrangement. The copper-binding site of APP is a rigid tetrahedral site, and Cu⁺ would be preferred. This site is also located on the surface of the molecule, which would leave the dangerous Cu⁺ exposed, and may explain some of the toxic effects observed with this domain.

### EXAMPLE 3

### Mutagenesis Data Supporting the His-His-Tyr-Met Binding Site

The NMR structure indicates that the copper-binding site is composed of His147, His151, Tyr168, Met170. Mutagenesis experiments were performed to test this. His147 and His151 were mutated to Asn, Tyr168 was mutated to Phe, and Met170 was mutated to Leu. These mutations were incorporated into APP133-189. Recombinant protein was expressed in Pichia pastoris as described above, and purified by standard chromatography methods. The identities of the purified proteins were verified by mass spectrometry. The Cu-reducing activity of the CuBD was tested in a lipid peroxidation assay.

Two different assays of metal mediated lipid peroxidation were utilized. The first assay involved measuring the oxidative activity of metallated proteins. This was determined by mixing dialyzed metallated or native protein at designated concentrations with 0.5 mg/mL low-density lipoprotein (LDL) for 24 hr (37° C.). Lipid peroxidation was measured using a lipid peroxidation assay kit (LPO 486, Oxis International Inc. Portland, Oreg.), as per kit instructions. The level of lipid peroxidation was determined by comparing absorbance (486 nm) with LDL alone (100% lipid peroxidation. The second assay was used to measure the lipid peroxidation activity of native proteins in the presence of free, non-protein-bound copper. This involved adding non-metallated peptides (140 μM) to 0.5 mg/mL LDL together with 20 μM Cu-glycine, and assaying for lipid peroxidation as for the metallated proteins. The level of lipid peroxidation was determined by comparing the absorbance (486 nm) with LDL+Cu-gly (100% lipid peroxidation). As a negative control, LDL was also exposed to dialysed Cu-glycine solutions comparable to those used to Cu-metallate the proteins.

The results are summarized in Table 2. The control is LDL (0.5 mg/mL) in PBS buffer alone. The APP28-123 is a negative control, and represents background activity. APP133-189 induced strong lipid peroxidation (3-fold above background). The His147Asn mutation abolished this activity, indicating that this histidine residue is critical for activity. The Met170Leu and Tyr168Phe mutations also had a significant effect on the activity of the CuBD. The His151Asn mutation also affected the activity, but to a lesser extent, indicating that His147 has a more important role than His151.

TABLE 2
LDL oxidation activity of Cu-metallated APP proteins

| | % of lipid |
|---|---|
| | peroxidation by |
| | control |
| Control | 100 |
| APP28-123 | 130 |
| APP133-189 | 312 |
| His147Asn | 87 |
| His151Asn | 196 |
| Tyr168Phe | 111 |
| Met170Leu | 142 |
| 100 μM Cu | 282 |

### EXAMPLE 4

### In Silico Docking

The chemical structure databases, Maybridge and Aldrich, were obtained from Tripos (Tripos Associates, 1699 S. Hanley Road, St. Louis, Mo. 63144, USA) in Unity format. These were converted to mol2 format using dbtranslate. software (Tripos). The National Cancer Institute (NCI) database was downloaded from the World-Wide Web (http://dtp.nci.nih.gov/docs/3d_database/structural inform ation/structural_data.html) in MDL sd format. This was converted to mol2 format using the program babel (BABEL-1.6. Walters, P. Stahl, M. (babel@mercury.aichem.arizona.edu)).

All databases were corrected for atom and bond types using in-house software. Entries which included atoms which were not recognisable in the scoring routines were removed. Entries with single heavy atoms (including carbon, nitrogen, oxygen, sulphur, and phosphorus) were removed. Protonation state and atom charges were assigned using the sybdb DOCK ancillary software (Kuntz, 1992). A short minimization of each ligand was performed to ensure good geometry.

The databases contain the following numbers of entries:

| | |
|---|---|
| Aldrich(1) | 58064 |
| Aldrich(2) | 106733 |
| Maybridge | 57114 |
| National Cancer Institue (NCI) | 115344 |

No attempt was made to ensure uniqueness between or within the databases. Some sites were docked to a reduced Aldrich database, in which the molecular weight was restricted to be less than 300 and the number of rotatable bonds to be less than 4 (Aldrich (1)).

Docking of entries from the databases to the protein sites was performed using the DOCK software suite. The energy grid used in DOCK was generated using the GRID software. A spacing of 0.25 Angstrom and an all-atom potential were used; apart from this, default parameters were used. Docking employed flexible ligands using the “torsion drive” option, with minimization. Potential sites for docking were initially identified by visual inspection using the InsightII software (Molecular Simulations Inc., San Diego, USA). Sites were further defined using ancillary DOCK software. All docking calculations were performed on a Beowulf cluster running the Linux operating system. Scheduling of tasks was performed using the Enfuzion software (TurboLinux Inc., 2000 Sierra Point Parkway, Suite 702, Brisbane, Calif. 94005, USA.). Scoring of individual DOCKed solutions was performed using in-house software. The scoring software included several scoring algorithms: Score (Wang et al, 1998), PMF (Muegge et al, 1999), ChemScore (Eldridge et al, 1997) and SmoG (DeWitte et al, 1996; 1997). Alternatives which involve subtle differences in these measures were also calculated, using Dock_Score Quality Estimate (Tao and Lai, 2001), and ChemScore with rotatable bond correction (Stahl and Rarey, 2001); SMoG score per heavy atom in the ligand and ligand intramolecular energies were also estimated (Baxter et al, 1998). Scoring was performed on the twenty-five solutions of lowest energy obtained from DOCK using a consensus measure (Wang and Wang, 2001).

From the four databases searched, 41 compounds showed a consensus score which was regarded as providing a potentially useful degree of fit. Representative structures thus identified are shown below.

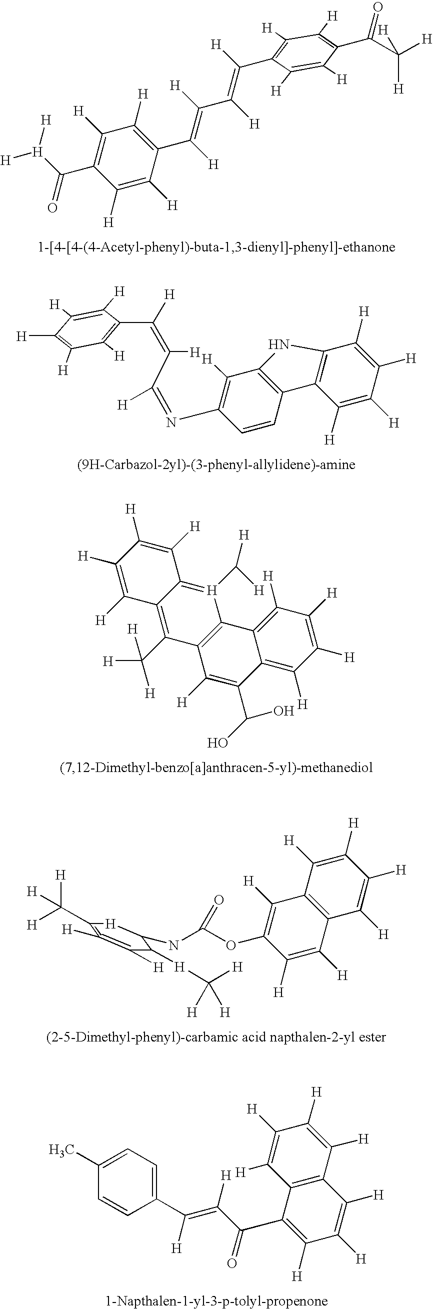

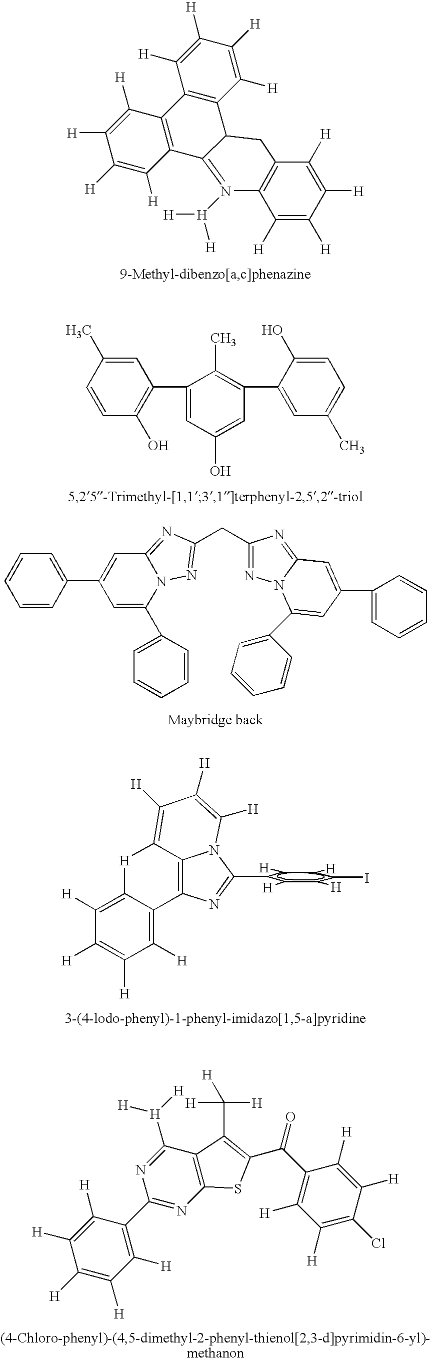

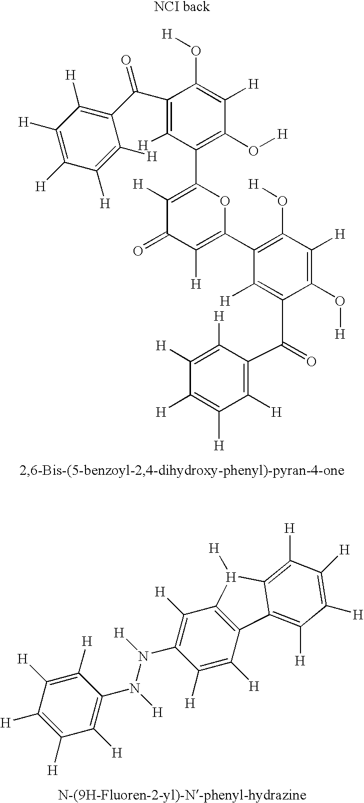

Of these, compounds 28879, 32499, 35548, 42623, 45007, and 48219 from the Aldrich database docked to the principal copper-binding site encompassing residues His 147, His 151, Tyr168 and Met 170 (identified as (a) herein), while Maybridge compounds 21056 and 54249, NCI compounds 31526 and 37643, and Aldrich compounds 76137 and 81972 docked to the secondary binding sites encompassing Leu136, Gln138, Glu139, Arg140, Met141, Val143, Glu145, His149, Trp150, Val153, Ala154, Thr157, Arg180, Gly181, Val182 and Phe184 or His147, His151, Tyr168, Met170, Ala154, Trp150, Lys155, Leu165, Phe179, Val182, and Glu183, identified herein as (b) and (c) respectively.

Docking of (2,5-dimethyl-phenyl)-carbamic acid napthalen-2-yl ester (Compound 42613) to the principal copper-binding site of APP is illustrated in FIG. 6, and docking of 3-(4-Iodo-phenyl)-1-phenyl-imidazo [1-5-a] pyridine (Compound 21056) to the secondary copper-binding site of APP which is located at the back of the copper-binding domain and encompasses the residues listed in (b) is shown in FIG. 7.

### EXAMPLE 5

### Cell Based Assay for Aβ Production

Aβ production is measured using either cell lines or primary cells. Cell lines can be either transfected with APP cDNA or untransfected. Cells are grown using suitable culture medium. The test compound is dissolved in an appropriate solvent, for example DMSO. The cells are treated with different concentrations of the test compound for different lengths of time. Aβ levels are measured by either western blotting or ELISA. The western blot assay involves separating either culture supernatant or cell lysate on SDS-PAGE, followed by western blotting to a membrane. The Aβ is detected using an antibody against Aβ. The levels of Aβ are quantitated by measuring the intensity of the Aβ signal or signals with a suitable detector, such as a phosphorimager or a densitometer. The change in Aβ level is determined by comparing to a non-drug treated control. An alternative method involves first immunoprecipitating the Aβ with an anti-Aβ antibody from either the culture supernatant or cell lysate prior to the SDS-PAGE step. The ELISA assay can be performed using a commercially-available ELISA kit (for example Human Amyloid Aβ (1-42) ELISA kit from IBL Hamburg, http://www.ibl-hamburg.com/prod/jp_{—}17711_amyloid-b-1-42.htm). The assay is performed in a ELISA plate which has been coated with an anti-Aβ antibody. The cell supernatant or cell lysate is added to the plate and incubated for a period of time. The plate is washed and then incubate with another anti-Aβ antibody. After a period of time the plate is washed. The amount of bound antibody may be measured using a reporter molecule that is coupled to the second antibody (for example horse radish peroxidase). The change in Aβ level is determined by comparing to a non-drug treated control.

### EXAMPLE 6

### Real Time Surface Plasmon Resonance Analysis

Real time binding experiments are performed on a BIACORE system equipped with the Upgrade kit (BIACORE, Pharmacia). All experiments are performed at 37° C. To prepare a metal chelating sensor surface, a nitrilotriacetic acid immobilized sensor ship (Sensor chip NTA, BIACORE, Uppsala, Sweden) is exposed to copper solution (100 μM CuCl₂ in Milli-Q water) for 4 min at a flow rate of 5 μl/min. For control experiments the sensor surface is treated as above, but by injecting EDTA (1 μM) for 4 min.

Surface Plasmon Resonance analysis (SPR) buffers and solutions are filtered and degassed: eluent buffer (PBS, 0.005% n-octylglycopyranoside, 1 μM EDTA, pH7.4), dispenser buffer (PBS, 0.005% n-octylglycopyranoside, 3 mM EDTA) and regeneration solutions I (50 mM EDTA) and II (45 mM EDTA, 1 mM bathocuproine disulphonate (BC). After extensive washing to reset the surface with regeneration buffer I followed by eluent buffer, individual flow cells are loaded with copper solution to saturate the surface with Cu(II). The signal for binding of Cu(II) to NTA is normally about 40 RU. Peptide stock solutions are prepared in 1 μM EDTA (1 mg/ml), diluted in PBS (30 μg/ml) and injected on to the surface for 2 min (10 μl) by using the KINJECT command. The sensorgram is allowed to run for an additional 20 min after the end of injection to determine the dissociation kinetics. Subsequent treatment with regeneration solution II for 6 min results in return to the baseline signal, indicating that the surface has completely been cleaned. Two observations are central to confirm the reliability of this approach. First, APP peptides do not show binding to the NTA surface when it has not previously been loaded with Cu(II). Second, the injection of 1 mM BC for 2 min on to the Cu(II)-charged NTA surface does not affect surface bound RU, showing that peptide binding is specific and exclusively mediated by Cu(II) but not Cu(I).

Sensorgrams are analyzed using the BIAevaluation 3.0 program (BIACORE) and kinetic constants are obtained by fitting curves to a single-site binding model (A+B=AB).

Using this method we have been able to demonstrate that APP124-189 is able to reduce Cu(II) to Cu(I), despite the fact that this fragment has no free cysteine residues.

Rational Drug Design

The process of rational drug design requires no explanation or teaching for the skilled person, but a brief description of computational design is given here for the lay reader. Various computational analyses are necessary to determine whether a molecule is sufficiently complementary to the target moiety or structure to be useful as a pharmaceutical agent. Some of these analyses are discussed above. Such analyses may be carried out in current software applications, such as the Molecular Similarity application of QUANTA (Molecular Simulations Inc., Waltham, Mass.) version 3.3, and as described in the accompanying User's Guide, Volume 3 pages 134-135.

The Molecular Similarity application permits comparisons between different structures, different conformations of the same structure, and different parts of the same structure. The procedure used in Molecular Similarity to compare structures is divided into four steps:

1) load the structures to be compared;

2) define the atom equivalences in these structures;

3) perform a fitting operation; and

4) analyze the results.

Each structure is identified by a name. One structure is identified as the target (ie., the fixed structure); all remaining structures are working structures (ie., moving structures). When a rigid fitting method is used, the working structure is translated and rotated to obtain an optimum fit with the target structure. The fitting operation uses a least squares fitting algorithm which computes the optimum translation and rotation to be applied to the moving structure, such that the root mean square difference of the fit over the specified pairs of equivalent atom is an absolute minimum. This number, given in angstroms, is reported by QUANTA.

One skilled in the art may use one of several methods to screen chemical entities or fragments for their ability to associate with a target site. Again, these methods require no elucidation for the skilled person, but are described here for the benefit of the unskilled reader. The screening process begins by visual inspection of the target site on the computer screen, generated from a machine-readable storage medium. Selected fragments or chemical entitles may then be positioned in a variety of orientations, or docked, within that binding pocket as defined above. Docking may be accomplished using software such as Quanta and Sybyl, followed by energy minimization and molecular dynamics with standard molecular mechanics force fields, such as CHARMM and AMBER.

Specialized computer programs may also assist in the process of selected fragments or chemical entities. These include:

1. GRID (Goodford, 1985). GRID is available from Oxford University, Oxford, UK.

2. MCSS (Miranker et al., 1991). MCSS is available from Molecular Simulations, Burlington, Mass.

3. AUTODOCK (Goodsell, 1990). AUTODOCK is available from Scripps Research Institute, La Jolla, Calif.

4. DOCK (Kuntz, 1982). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target compound or site. This would be followed by manual model building using software such as Quanta or Sybyl.

Useful programs to aid one of skill in the art in connecting the individual chemical entities or fragments include:

1. CAVEAT (Bartlett, 1989). CAVEAT is available from the University of California, Berkeley, Calif.

2. 3D Database systems such as MACCS-3D (MDL Information Systems, San Leandro, Calif.). This area is reviewed by Martin (1992).

3. HOOK (available from Molecular Simulations Burlington, Mass.).

As the skilled reader will already know, instead of proceeding to build a ligand for the target in a step-wise fashion, one fragment or chemical entity at a time as described above, target-binding compounds may be designed as a whole or de novo. These methods include:

1. LUDI (Bohm, 1992). LUDI is available from the Biosym Technologies, San Diego, Calif.

2. LEGEND (Nishibata, 1991). LEGEND is available from Molecular Simulations, Burlington, Mass.

3. LeapFrog (available from Tripos Associates, St. Louis. Mo.).

Other molecular modelling techniques may also be employed. See for example Cohen (1990). See also Navia (1992).

Once a compound has been designed or selected by such methods, the efficiency with which that compound can bind to a target site may be tested and optimized by computational evaluation. For example, an effective ligand will preferably demonstrate a relatively small difference in energy between its bound and free states, ie. a small deformation energy of binding. Thus the most efficient ligand should preferably be designed with a deformation energy of binding of not greater than about 10 kcal/mole, preferably, not greater than 7 kcal/mole. Ligands may interact with the target in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a target may be further computionally optimized so that in its bound states it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the ligand and the target, when the ligand is bound to the target, preferably makes a neutral or favourable contribution to the enthalpy of binding.

Specific computer software is available in the art to evaluate compound deformation energy and electrostatic interaction. Examples of programs designed for such uses include: Gaussian 92, revision C [M. J. Frisch, Gaussian, Inc., Pittsburgh, Pa. COPYRGT. 1992]; AMBER, version 4.0 [P.A. Kollman, University of California at San Francisco, COPYRGT. 1994]; QUANTA/CHARMM [Molecular Simulations, Inc., Burlington, Mass. COPYRGT. 1994]; and Insight II/Discover (Biosysm Technologies Inc., San Diego, Calif. COPYRGT. 1994). These programs may be implemented, for instance, using a Silicon Graphics workstation, IRIS 4D/35 or IBM RISC/6000 workstation model 550. Other hardware systems and software packages will be known to those skilled in the art.

Once the ligand has been optimally selected or designed, as described above, substitutions may then be made in some of its atoms or side groups in order to improve or modify its binding properties. Generally initial substitutions are conservative, ie., the replacement group will have approximately the same size, shape, hydrophobicity and charge as the original group. It should, of course, be understood that components known in the art to alter conformation should be avoided. Such substituted chemical compounds may then be analyzed for efficiency of fit to the desired target site by the same computer methods described in detail, above. Again, all these facts are familiar to the skilled person.

Another approach is the computational screening of small molecule data bases for chemical entities or compounds which can bind in whole, or in part, to a desired target. In this screening, the quality of fit of such entities to the binding site may be judged either by shape complementarity or by estimated interaction energy. (Meng, 1992).

The computational analysis and design of molecules, as well as software and computer systems therefor, are described in U.S. Pat. No. 5,978,740 which is included herein by reference, including specifically but not by way of limitation the computer system diagram described with reference to and illustrated in FIG. 3 thereof, as well as the data storage media diagram described with reference to and illustrated in FIGS. 4s and 5 thereof.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Bartlett, P. A. CAVEAT: A Program to Facilitate the Structure-Derived Design of Biologically Active Molecules. In Molecular Recognition in Chemical and Biological Problems. Special Pub., Royal Chem. Soc., 78 pp. 182-196 (1989)
Baxter, C. A., Murray, C. W., Clark, D. E., Westhead, D. R., Eldridge, M. D. Flexible docking using tabu search and an empirical estimate of binding affinity. Proteins 33: 367-382, 1998.
Beddell, Drug development based on information regarding receptor structure. Chem. Soc. Reviews 1985, 279;
Blundell et al., Nature 1987 326 347
Bohm H.-J., The Computer Program LUDI: A New Method for the De Novo Design of Enzyme Inhibitors. J. Comp. Aid. Molec. Design 6, pp. 61-78 (1992)
Borchardt T., Camakaris J., Cappai R., Masters C. L., Beyreuther K. and Multhaup G. (1999). Copper inhibits beta-amyloid production and stimulates the non-amyloidogenic pathway of amyloid-precursor-protein secretion. Biochem J. 344: 461-7.
Brunger, A. T., Acta Crystallogr. D. Biol Crystallogr 54: 905-21 (1988).
Bush, A. I Multhaup, G., Moir, R. D., Williamson, T. G., Small, D. H., Rumble, B., Pollweinn, P., Beyreuther, K. and Masters. C. L. A Novel Zinc (II) Binding Site Modulates the Function of the βA4 Amyloid Protein Precursor of Alzheimer's Disease, Journal of Biological Chemistry 1993) 268, 16109-16112
Bush A. I. (2000) Metals and neuroscience. Curr Opin Chem Biol 4, 184-191.
Cohen, N. C. et al. Molecular Modeling Software and Methods for Medicinal Chemistry, J. Med. Chem., 33, pp. 883-894′ (1990)
Day C. L., Dupont C., Lackman, M., Vaux D. L. and Hinds M. G (1999) Cell Death and Diff., 6, 1125-1132.
DeWitte, R. S., Shakhnovich, E. I. SMoG: de novo design method based on simple fast and accurate free energy estimates. 1. Methodology and supporting evidence. J. Am. Chem. Soc. 118: 11733-11744, 1996.
De Witte, R. S., Ischenko, A. V., Shakhnovich, E. I. SMoG. de novo design method based on simple fast and accurate free energy estimates. 2. Case studies in molecular design. J. Am. Chem. Soc. 119:4608-4617, 1997.
Eldridge, M. D., Murray, C. W., Auton, T. R., Paolini, G. V., Mee, R. P. Empirical scorint functions:I. The development of a fast empirical scoring function to estimate the binding affinity of ligands in receptor complexes. J. Comp.-Aided Mol. Des. 11: 425-445, 1997.
Glenner G. G. and Wong C. W. 1984. Alzheimer's disease: initial report of the purification and characterization of a novel cerebovascular amyloid protein. Biochem. Biophys. Res. Commun. 120: 885-890.
Goodford, J. Med. Chem. 1984 27 557;
Goodford, P. J. A Computational Procedure for Determining Energetically Favorable Binding Sites on Biologically Important Macromolecules. J. Med. Chem., 28, pp. 849-857 (1985)
Goodsell, D. S. et al. Automated Docking of Substrates to Proteins by Simulated Annealing. Proteins: Structure, Function, and Genetics, 8, pp. 195-202 (1990)
Hardy J. Trends in Neuroscience (1987) 20:154-159.
Henry A., Masters C. L., Beyreuther K. and Cappai R. (1997). “Expression of the ectodomains of the human amyloid precursor protein in Pichia pastoris: Analysis of culture conditions, purification, and characterization.” Protein. Expr and Purif 10: 283-291.
Hol, Angew. Chem. 1986 25 767
Holmgren A., Soderberg B.-O., Eklund H. and Branden C.-I. (1975) Proc. Natl. Acad. Sci. USA. 72, 2305
Ito N., Phillips S. E. V., Stevens C., Ogel Z. B., McPherson M. J., Keen J. N., Yadav K. D. S. and Knowles P. F. (1991) Nature 350, 87
Kang J, Lemaire H., Unterbeck A., Salbaum J. M., Masters C. L., Grzeschik K., Multhaup G., Beyreuther K. and Müller-Hill B. (1987) The precursor of Alzheimer's disease amyloid A4 protein resembles a cell-surface receptor. Nature 325: 733-736.
Karlin S., Zhu Z.-Y. and Karlin K. D. (1997) Proc. Natl. Acad. Sci. USA. 94, 14225-14230.
Klaus W., Broger C., Gerber P. and Senn N. (1993) Determination of the disulfide bonding pattern in proteins by local and global analysis of nuclear magnetic resonance data. J. Mol. Biol. 232, 897-906.
Kuntz, I. D. et al. A Geometric Approach to Macromolecule-Ligand Interactions. J. Mol. Biol. 161, pp. 269-288 (1982)
Kuntz, I. D. Structure-based strategies for drug design and discovery. Science 257: 1078-1082, 1992.
Laroche Y., Storme V., De Meutter J., Messens J. and Lauwereys M. (1994). High-level secretion and very efficient isotopic labeling of tick anticoagulant peptide (TAP) expressed in the methylotrophic yeast, Pichia pastoris. Biotechnology 12: 1119-24.
Ludvigsen S. and Poulsen F. M. (1992) Positive □-angles in proteins by nuclear magnetic resonance and distance geometry. J. Biomol. NMR 2, 227-233.
Martin Y. C., 3D Database Searching in Drug Design. J. Med. Chem., 35, pp. 2145-2154 (1992)
Meng, E. C. et al. J. Comp. Chem., 13, pp. 505-524 (1992)
Miranker A. et al. Functionality Maps of Binding Sites: A Multiple Copy Simultaneous Search Method. Proteins: Structure, Function and Genetics, 11, pp. 29-34 (1991)
Muegge, I., Martin, Y. C. A general and fast scoring function for proten-ligand interactios: A simplified potential approach. J. Med. Chem. 42:791-804, 1999.
Multhaup G., Ruppert T., Schlicksupp A., Hesse L., Eckhard E., Pipkorn R., Masters C. L. and Beyreuther K. (1998). Copper-binding amyloid precursor protein undergoes a site-specific fragmentation in the reduction of hydrogen peroxide. Biochemistry 37: 7224-7230.
Navia, M. A. et al. The Use of Structural Information in Drug Design, Current Opinions in Structural Biology, 2, pp. 202-210 (1992)
Nishibata Y. et al., Tetrahedron, 47, p. 8985 (1991)
Prigge S. T., Kolhekar A. S., Eipper B. A. and Mains R. E., (1997) Science, 278 1300.
Rossjohn, J., Cappai, R., Feil, S. C., Henry, A., McKinstry, W. J., Galatis, D., Hesse, L., Multhaup, G., Beyreuther, K., Masters, C. L. and Parker, M. W. Crystal structure of the N-terminal, growth factor-like domain of Alzheimer amyloid precursor protein. Nature Structural Biology. (1999) 6, 327-331
Sattler M., Schlencher J., and Griesinger C. (1999) Prog. NMR Spect. 34, 93-158.
Sheridan and Venkataraghavan, Acc. Chem Res. 1987 20 322;
Stahl, M., Rarey, M. Detailed analysis of scoring functions for virtual screening. J. Med. Chem. 2001.
Tao, P. Lai, L. Protein ligand docking based on empirical method for binding affinity estimiation. J. Comp.-Aided Mol. Des. 15:429-446, 2001.
Verlinde C. L. M. J & Hol, W. G. J. Structure 1994, 2, 577
Wang, R., Lui, L., Lai, L., Tang, Y. SCORE: A new empirical method for estimating the binding affinity of a protein-ligand complex. J. Mol. Model. 4: 379-394, 1998.
Wang, R., Wang, S. How does consensus scoring work for virtual library screening? An idealized computer experiment. J. Chem. Inf. Comput. Sci. 2001.
White, A. R., Multhaup G., Maher, F., Bellingham, S., Camakaris, J., Zheng, H., Bush, A. I., Beyreuther, K., Masters, C. L., Cappai, R., (1999a). The Alzheimer's disease amyloid precursor protein modulates copper-induced toxicity and oxidative stress in primary neuronal cultures. J. Neurosci 19:9170-9179.
White, A. R., Mercer, J. F. B., Camakaris, J., Zheng, H., Bush, A. I., Multhaup G., Beyreuther, K., Masters, C. L., Cappai, R. (1999b) Copper levels are increased in the cerebral cortex and liver of APP and APLP2 knockout mice. Brain Res. 842:439-444.

Yanker B. A, (1996) Mechanisms of neuronal degeneration in Alzheimer's disease. Neuron. 16, 921-932

LENGTHY TABLE REFERENCED HERE
US20070015688A1-20070118-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070015688A1) An electronic copy of the table will also be available from the USPTO upon request and payment of the fee set forth in 37 CFR 1.19(b)(3).

1. A method of identifying a compound which is capable of acting as an agonist of binding of divalent copper ions to APP, comprising the step of identifying a compound which has a conformation and polarity such that it interacts with an amino acid residue in APP selected from the group consisting of:
a) His147, His151, Tyr168 and Met170;
b) Leu136, Gln138, Glu139, Arg140, Met141, Val143, Glu145, His149, Trp150, Val153, Ala154, Thr157, Arg180, Gly181, Val182 and Phe184; and
c) His147, His151, Tyr168, Met170, Ala154, Trp150, Lys155, Leu165, Gly169, Phe179, Val182, Glu183 and Phe184

2. A method according to claim 1, in which the compound has the ability to interact with the copper-binding domain of APP in such a way that the extent of processing of APP to Aβ or to Aβ-containing fragments in the presence of the compound is reduced, compared to that of APP in the absence of the compound.

3. A method according to claim 1 or claim 2, in which the compound binds to at least two of the amino acids set out in (a), (b) or (c).

4. A method according to claim 1 or claim 2, in which the compound binds to at least three of the amino acids set out in (a), (b) or (c).

5. A method according to claim 1 or claim 2, in which the compound binds to four of the amino acids set out in (a), (b) or (c).

6. A method according to any one of claims 1 to 5, in which the compound has the ability to penetrate the blood-brain barrier.

7. A method according to any one of claims 1 to 6, in which the compound has the effect of stabilising binding of copper to the copper-binding site, and the compound stabilises the oxidation state of the copper, thereby inhibiting production of toxic Cu(I) ions.

8. A method according to any one of claims 1 to 5, in which the compound interacts directly with one or more amino acids selected from the group consisting of His147, His151, Tyr168 and Met170.

9. A method according to claim 8, in which the compound is a metal complex which can bind to the imidazole of a histidine residue.

10. A computer-assisted method for identifying compounds potentially able to bind to the copper-binding domain of the APP and decrease the processing of the APP into Aβ or Aβ containing fragments, using a programmed computer comprising the steps of:
(a) inputting into the programmed computer data comprising the atomic coordinates of the APP copper-binding domain, as shown in Appendix A, corresponding to the binding site defined by residues
(i) His147, His151 and Tyr168 and Met170
(ii) Leu136, Gln138, Glu139, Arg140, Met141, Val143, Glu145, His149, Trp150, Val153, Ala154, Thr157, Arg180, Gly181, Val182 and Phe184, or
(iii) His147, His151, Tyr168, Met170, Ala154, Trp150, Lys155, Leu165, Gly169, Phe179, Val182, Glu183 and Phe184,
or a subset thereof;
(b) generating, using computer methods, a set of atomic coordinates of a structure that possesses stereochemical complementarity to the atomic coordinates defined in (a) or a subset thereof, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting the selected chemical structures which are similar to a portion of the criteria data set.

11. A method according to claim 10, in which the method further comprises the step of obtaining a compound with a chemical structure selected in steps (d) and (e), and testing the compound for the ability to decrease processing of the APP into Aβ or Aβ-containing fragments.

12. A computer or a software component thereof for producing a three-dimensional representation of a molecule or molecular complex, which comprises a three-dimensional representation of a homologue of the molecule or molecular complex, in which the homologue comprises a binding domain that has a root mean square deviation from the backbone atoms of the amino acids of not more than 1.5 Å, in which the computer comprises:
(a) a machine-readable data storage medium comprising a data storage material encoded with machine-readable data, wherein the data comprises the structure coordinates, as shown in Appendix A, of:
(i) His147, His151 and Tyr168 and Met170
(ii) Leu136, Gln138, Glu139, Arg140, Met141, Val143, Glu145, His149, Trp150, Val153, Ala154, Thr157, Arg180, Gly181, Val182 and Phe184, or
(iii) His147, His151, Tyr168, Met170, Ala154, Trp150, Lys155, Leu165, Gly169, Phe179, Val182, Glu183 and Phe184;
(b) a working memory for storing instructions for processing the machine-readable data;
(c) a central-processing unit coupled to the working memory and to the machine-readable data storage medium for processing the machine-readable data into the three-dimensional representation; and
(d) a display coupled to the central-processing unit for displaying the three-dimensional representation.

13. A computer or a software component thereof for determining at least a portion of the structure coordinates corresponding to a three-dimensional structure of a molecule or molecular complex, in which the computer comprises:
(a) a machine-readable data storage medium comprising a data storage material encoded with machine-readable data, in which the data comprises at least a portion of the structural coordinates according to Appendix A;
(b) a machine-readable data storage medium comprising a data storage material encoded with machine-readable data, wherein the data comprise the NMR structural coordinates of the molecule or molecular complex;
(c) a working memory for storing instructions for processing the machine-readable data of (a) and (b);
(d) a central-processing unit coupled to the working memory and to the machine-readable data storage medium of (a) and (b) for performing a Fourier transform to the machine readable data of (a) and for processing the machine-readable data of (b) into structure coordinates; and
(e) a display coupled to the central-processing unit for displaying the structure coordinates of the molecule or molecular complex.

14. A compound able to act as an agonist of the binding of copper to the copper-binding domain of APP, wherein the compound is obtained by a method according to any one of claims 1 to 11.

15. A pharmaceutical composition comprising a compound according to claim 14, together with a pharmaceutically-acceptable carrier.

16. A composition according to claim 15, in which the compound is conjugated to a targeting moiety.

17. A composition according to claim 16, in which the ligand-targeting moiety complex is able to pass through the blood-brain barrier.

18. A method of reducing the processing of APP into Aβ or Aβ containing fragments, comprising the steps of exposing the APP to a composition according to any one of claims 14 to 16.

19. A method of reducing the amyloidogenic processing of APP, comprising the steps of exposing APP to a composition according to any one of claims 15 to 17.

20. A method of treating Alzheimer's disease or other amyloid-related condition, the method comprising administering to a subject in need thereof a composition according to any one of claims 15 to 17.

21. A method according to any one of claims 18 to 20, in which the compound has a conformation and polarity such that it binds to at least one amino acid residue in APP selected from the group consisting of:
a) His147, His151 and Tyr168 and Met170;
b) Leu136, Gln138, Glu139, Arg140, Met141, Val143, Glu145, His149, Trp150, Val153, Ala154, Thr157, Arg180, Gly181, Val182 and Phe184; and
c) His147, His151, Tyr168, Met170, Ala154, Trp150, Lys155, Leu165, Gly169, Phe179, Val182, Glu183 and Phe184.

22. A method according to claim 21, in which the compound binds to at least two of the amino acids set out in (a), (b) or (c).

23. A method according to claim 22, in which the compound binds to at least three of the amino acids set out in (a), (b) or (c).

24. A method according to claim 23, in which the compound binds to four of the amino acids set out in (a), (b) or (c).


SEQUENCE LISTING (3 sequences)

SEQ ID NO: 1 - protein, 66 aa, Homo sapiens
SDALLVPDKCKFLHQERMDVCETHLHWHTVAKETCSEKSTNLHDYGMLLPCGIDKFRGVEFVCCPL

SEQ ID NO: 2 - DNA, 35 bp, Artificial
  DNA primer
gctcgagaaaagagaggctagtgatgcccttctcg

SEQ ID NO: 3 - DNA, 30 bp, Artificial
  DNA primer
gaattcttacagtgggcaacacacaaactc